SEQUENCE LISTING (15 sequences)

SEQ ID NO: 1 - DNA, 2467 bp, Burkhorderia cepacia
  CDS (258)..(761)
  CDS (764)..(2380)
  CDS (2386)..(2466)
aagctttctgtttgattgcacgcgattctaaccgagcgtctgtgaggcggaacgcgacatgcttcgtgtcgcacacgtgtcgcgccgacgacacaaaaatgcagcgaaatggctgatcgttacgaatggctgacacattgaatggactataaaaccattgtccgttccggaatgtgcgcgtacatttcaggtccgcgccgatttttgagaaatatcaagcgtggttttcccgaatccggtgttcgagagaaggaaacatgcacaacgacaacactccccactcgcgtcgccacggcgacgcagccgcatcaggcatcacgcggcgtcaatggttgcaaggcgcgctggcgctgaccgcagcgggcctcacgggttcgctgacattgcgggcgcttgcagacaaccccggcactgcgccgctcgatacgttcatgacgctttccgaatcgctgaccggcaagaaagggctcagccgcgtgatcggcgagcgcctgctgcaggcgctgcagaagggctcgttcaagacggccgacagcctgccgcagctcgccggcgcgctcgcgtccggttcgctgacgcctgaacaggaatcgctcgcactgacgatcctcgaggcctggtatctcggcatcgtcgacaacgtcgtgattacgtacgaggaagcattaatgttcggcgtcgtgtccgatacgctcgtgatccgttcgtattgccccaacaaacccggcttctgggccgacaaaccgatcgagaggcaagcctgatggccgataccgatacgcaaaaggccgacgtcgtcgtcgttggatcgggtgtcgcgggcgcgatcgtcgcgcatcagctcgcgatggcgggcaaggcggtgatcctgctcgaagcgggcccgcgcatgccgcgctgggaaatcgtcgagcgcttccgcaatcagcccgacaagatggacttcatggcgccgtacccgtcgagcccctgggcgccgcatcccgagtacggcccgccgaacgactacctgatcctgaagggcgagcacaagttcaactcgcagtacatccgcgcggtgggcggcacgacgtggcactgggccgcgtcggcgtggcgcttcattccgaacgacttcaagatgaagagcgtgtacggcgtcggccgcgactggccgatccagtacgacgatctcgagccgtactatcagcgcgcggaggaagagctcggcgtgtggggcccgggccccgaggaagatctgtactcgccgcgcaagcagccgtatccgatgccgccgctgccgttgtcgttcaacgagcagaccatcaagacggcgctgaacaactacgatccgaagttccatgtcgtgaccgagccggtcgcgcgcaacagccgcccgtacgacggccgcccgacttgttgcggcaacaacaactgcatgccgatctgcccgatcggcgcgatgtacaacggcatcgtgcacgtcgagaaggccgaacgcgccggcgcgaagctgatcgagaacgcggtcgtctacaagctcgagacgggcccggacaagcgcatcgtcgcggcgctctacaaggacaagacgggcgccgagcatcgcgtcgaaggcaagtatttcgtgctcgccgcgaacggcatcgagacgccgaagatcctgctgatgtccgcgaaccgcgatttcccgaacggtgtcgcgaacagctcggacatggtcggccgcaacctgatggaccatccgggcaccggcgtgtcgttctatgcgagcgagaagctgtggccgggccgcggcccgcaggagatgacgtcgctgatcggtttccgcgacggtccgttccgcgcgaccgaagcggcgaagaagatccacctgtcgaacctgtcgcgcatcgaccaggagacgcagaagatcttcaaggccggcaagctgatgaagcccgacgagctcgacgcgcagatccgcgaccgttccgcacgctacgtgcagttcgactgcttccacgaaatcctgccgcaacccgagaaccgcatcgtgccgagcaagacggcgaccgatgcgatcggcattccgcgccccgagatcacgtatgcgatcgacgactacgtgaagcgcggcgccgcgcatacgcgcgaggtctacgcgaccgccgcgaaggtgctcggcggcacggacgtcgtgttcaacgacgaattcgcgccgaacaatcacatcacgggctcgacgatcatgggcgccgatgcgcgcgactccgtcgtcgacaaggactgccgcacgttcgaccatccgaacctgttcatttcgagcagcgcgacgatgccgaccgtcggtaccgtaaacgtgacgctgacgatcgccgcgctcgcgctgcggatgtcggacacgctgaagaaggaagtctgaccgtgcggaaatctactctcactttcctcatcgccggctgcctcgcgttgccgggcttcgcgcgcgcggccgatgcggccgatc

SEQ ID NO: 2 - protein, 168 aa, Burkhorderia cepacia
MHNDNTPHSRRHGDAAASGITRRQWLQGALALTAAGLTGSLTLRALADNPGTAPLDTFMTLSESLTGKKGLSRVIGERLLQALQKGSFKTADSLPQLAGALASGSLTPEQESLALTILEAWYLGIVDNVVITYEEALMFGVVSDTLVIRSYCPNKPGFWADKPIERQA

SEQ ID NO: 3 - protein, 539 aa, Burkhorderia cepacia
MADTDTQKADVVVVGSGVAGAIVAHQLAMAGKAVILLEAGPRMPRWEIVERFRNQPDKMDFMAPYPSSPWAPHPEYGPPNDYLILKGEHKFNSQYIRAVGGTTWHWAASAWRFIPNDFKMKSVYGVGRDWPIQYDDLEPYYQRAEEELGVWGPGPEEDLYSPRKQPYPMPPLPLSFNEQTIKTALNNYDPKFHVVTEPVARNSRPYDGRPTCCGNNNCMPICPIGAMYNGIVHVEKAERAGAKLIENAVVYKLETGPDKRIVAALYKDKTGAEHRVEGKYFVLAANGIETPKILLMSANRDFPNGVANSSDMVGRNLMDHPGTGVSFYASEKLWPGRGPQEMTSLIGFRDGPFRATEAAKKIHLSNLSRIDQETQKIFKAGKLMKPDELDAQIRDRSARYVQFDCFHEILPQPENRIVPSKTATDAIGIPRPEITYAIDDYVKRGAAHTREVYATAAKVLGGTDVVFNDEFAPNNHITGSTIMGADARDSVVDKDCRTFDHPNLFISSSATMPTVGTVNVTLTIAALALRMSDTLKKEV

SEQ ID NO: 4 - protein, 27 aa, Burkhorderia cepacia
VRKSTLTFLIAGCLALPGFARAADAAD

SEQ ID NO: 5 - protein, 16 aa, Burkhorderia cepacia
ADAADPALVKRGEYLA

SEQ ID NO: 6 - protein, 25 aa, Artificial Sequence
  Description of Artificial Sequenceconsensus
ADAADXALVKRGEYLAXXXDCXACH

SEQ ID NO: 7 - DNA, 27 bp, Artificial Sequence
  Description of Artificial Sequence primer
tgcaccgtgcggaaatctactctcact

SEQ ID NO: 8 - DNA, 27 bp, Artificial Sequence
  Description of Artificial Sequence primer
acttccttcttcagcgtgtccgacatc

SEQ ID NO: 9 - DNA, 1441 bp, Burkholderia cepacia
  CDS (121)..(1398)
tccgaacctgttcatttcgagcagcgcgacgatgccgaccgtcggtaccgtaaacgtgacgctgacgatcgccgcgctcgcgctgcggatgtcggacacgctgaagaaggaagtctgaccgtgcggaaatctactctcactttcctcatcgccggctgcctcgcgttgccgggcttcgcgcgcgcggccgatgcggccgatccggcgctggtcaagcgcggcgaatacctcgcgaccgccatgccggtaccgatgctcggcaagatctacacgagcaacatcacgcccgatcccgatacgggcgactgcatggcctgccacaccgtgaagggcggcaagccgtacgcgggcggccttggcggcatcggcaaatggacgttcgaggacttcgagcgcgcggtgcggcacggcgtgtcgaagaacggcgacaacctgtatccggcgatgccgtacgtgtcgtacgcgaagatcaaggacgacgacgtacgcgcgctgtacgcctacttcatgcacggcgtcgagccggtcaagcaggcgccgccgaagaacgagatcccagcgctgctaagcatgcgctggccgctgaagatctggaactggctgttcctgaaggacggcccgtaccagccgaagccgtcgcagagcgccgaatggaatcgcggcgcgtatctggtgcagggtctcgcgcactgcagcacgtgccacacgccgcgcggcatcgcgatgcaggagaagtcgctcgacgaaaccggcggcagcttcctcgcggggtcggtgctcgccggctgggacggctacaacatcacgtcggacccgaatgcggggatcggcagctggacgcagcagcagctcgtgcagtatttgcgcaccggcagcgtgccgggcgtcgcgcaggcggccgggccgatggccgaggcggtcgagcacagcttctcgaagatgaccgaagcggacatcggtgcgatcgccacgtacgtccgcacggtgccggccgttgccgacagcaacgcgaagcagccgcggtcgtcgtggggcaagccggccgaggacgggctgaagctgcgcggtgtcgcgctcgcgtcgtcgggcatcgatccggcgcggctgtatctcggcaactgcgcgacgtgccaccagatgcagggcaagggcacgccggacggctattacccgtcgctgttccacaactccaccgtcggcgcgtcgaatccgtcgaacctcgtgcaggtgatcctgaacggcgtgcagcgcaagatcggcagcgaggatatcgggatgcccgctttccgctacgatctgaacgacgcgcagatcgccgcgctgacgaactacgtgaccgcgcagttcggcaatccggcggcgaaggtgacggagcaggacgtcgcgaagctgcgctgacatagtcgggcgcgccgacacggcgcaaccgataggacaggag

SEQ ID NO: 10 - protein, 425 aa, Burkholderia cepacia
VRKSTLTFLIAGCLALPGFARAADAADPALVKRGEYLATAMPVPMLGKIYTSNITPDPDTGDCMACHTVKGGKPYAGGLGGIGKWTFEDFERAVRHGVSKNGDNLYPAMPYVSYAKIKDDDVRALYAYFMHGVEPVKQAPPKNEIPALLSMRWPLKIWNWLFLKDGPYQPKPSQSAEWNRGAYLVQGLAHCSTCHTPRGIAMQEKSLDETGGSFLAGSVLAGWDGYNITSDPNAGIGSWTQQQLVQYLRTGSVPGVAQAAGPMAEAVEHSFSKMTEADIGAIATYVRTVPAVADSNAKQPRSSWGKPAEDGLKLRGVALASSGIDPARLYLGNCATCHQMQGKGTPDGYYPSLFHNSTVGASNPSNLVQVILNGVQRKIGSEDIGMPAFRYDLNDAQIAALTNYVTAQFGNPAAKVTEQDVAKLR

SEQ ID NO: 11 - protein, 5 aa, Artificial Sequence
  Description of Artificial Sequence heme binding motif
CXXCH

SEQ ID NO: 12 - DNA, 26 bp, Artificial Sequence
  Description of Artificial Sequence primer
catgccatggcacacaacgacaacac

SEQ ID NO: 13 - DNA, 30 bp, Artificial Sequence
  Description of Artificial Sequence primer
gtcgacgatcttcttccagccgaacatcac

SEQ ID NO: 14 - DNA, 30 bp, Artificial Sequence
  Description of Artificial Sequence primer
tggccatggttgaagccagagagttacttt

SEQ ID NO: 15 - DNA, 30 bp, Artificial Sequence
  Description of Artificial Sequence primer
ttatttactctcctgcggcgacaaatgttg

(19) United States
(12) Patent Application Publication
(10) Pub. No.: US 2006/0194278 A1
(43) Pub. Date: Aug. 31, 2006

(54) Process for producing glucose dehydrogenase

(76) Inventors: Hideaki Yamaoka, Kyoto-shi (JP); Mitsuhiro Hoshijima, Kyoto-shi (JP); Shido Kawase, Uji-shi (JP); Keisuke Kurosaka, Uji-shi (JP)

Correspondence Address: KNOBBE MARTENS OLSON & BEAR LLP, 2040 MAIN STREET, FOURTEENTH FLOOR, IRVINE, CA, 92614, US

(21) Appl. No.: 10/550,671

(22) PCT Filed: Mar. 24, 2004
(86) PCT No.: PCT/JP04/04074

§ 371 Date: Nov. 9, 2005

(30) Foreign Application Priority Data
Mar. 25, 2003 (JP) 2003-082739

(51) Int. Cl. C12P 21/06; C12N 9/02; C12N 1/21; C12N 15/74

(52) U.S. Cl. 435/69.1; 435/189; 435/252.33; 435/488

(57) ABSTRACT

A glucose dehydrogenase complex is produced by culturing an Escherichia bacterium, which is introduced with DNAs encoding the α-subunit and the β-subunit of glucose dehydrogenase of Burkhorderia cepacia in an expressible form, and in which expression of the ccm system (cytochrome c maturation system) is enhanced, to express the aforementioned DNAs and produce a glucose dehydrogenase complex, and collecting the complex.

### TECHNICAL FIELD

The present invention relates to a method for producing an Escherichia bacterium that abundantly expresses a glucose dehydrogenase complex of Burkhorderia cepacia and a method for producing the enzyme complex. Glucose dehydrogenase is useful in a glucose sensor using an enzyme electrode and so forth.

### BACKGROUND ART

As a glucose dehydrogenase (GDH), an enzyme produced by a microorganism belonging to the genus Burkhorderia (Burkhorderia cepacia KS1 strain) has been known. This enzyme is a complex consisting of the α-subunit, which is a catalyst subunit, the β-subunit, which is cytochrome c, and the γ-subunit, and has superior properties that it has high thermal stability, the reaction catalyzed by it is hardly affected by dissolved oxygen, and so forth. DNAs encoding the α-subunit and the γ-subunit of this enzyme have been isolated, and a part of a DNA encoding the β-subunit has also been isolated. Furthermore, this enzyme has been successfully expressed in Escherichia coli (refer to International Patent Publication WO02/36779). However, the expressed GDH does not appear to contain the β-subunit.

Meanwhile, as a cytochrome maturation system of Escherichia coli, the ccm system (cytochrome c maturation system) is known. The ccm system is known to be expressed in Escherichia coli under anaerobic and special conditions (J. Bacteriol. 177, 4321-4326 (1995)). The DNA sequence of the operon encoding the ccm system (ccmABCDEFGH) has already been elucidated (Nature, 409 (6819), 529-533 (2001); GeneBank database accession U0008 (1993)). Furthermore, it has been reported that a covalently bonded type multi-heme cytochrome derived from an organism of a species different from Escherichia coli was expressed and produced under an aerobic condition in Escherichia coli transformed with a plasmid that expresses the ccm system (Biochem. Biophys. Res. Commun., 251, 744-7 (1998); Biochem. Biophys. Acta, 1411, 114-20 (1999); Biochem. Biophys. Acta, 1481, 18-24 (2000); Protein Sci. 9, 2074-84 (2000)).

Furthermore, as a plasmid containing the ccm operon, the plasmid pEC86 obtained by inserting the operon into pACYC184 is known (Biochem. Biophys. Res. Commun., 251, 744-7 (1998)).

### DISCLOSURE OF THE INVENTION

The inventors of the present invention previously found that the expression efficiency was lower when the α-subunit and the β-subunit of the aforementioned glucose dehydrogenase were simultaneously expressed in Escherichia coli than when the α-subunit alone was expressed. Thus, an object of the present invention is to provide a means for abundantly expressing an enzyme complex containing the α-subunit and the β-subunit in an Escherichia bacterium.

The inventors of the present invention assiduously studied in order to achieve the foregoing object. As a result, they found that expression of a DNA encoding a glucose dehydrogenase complex of Burkhorderia cepacia could be improved in an Escherichia bacterium by enhancing the expression of the ccm system of the bacterium, and thus accomplished the present invention.

The present invention thus provides the followings.

(1) An Escherichia bacterium, which is introduced with DNAs encoding the α-subunit and the β-subunit of glucose dehydrogenase of Burkhorderia cepacia in an expressible form, and wherein expression of the ccm system is enhanced.

(2) The Escherichia bacterium according to (1), wherein the DNA encoding the α-subunit locates upstream from the DNA encoding the β-subunit, and expressions of them are regulated by a single promoter.

(3) The Escherichia bacterium according to (1), which is further introduced with a DNA encoding the γ-subunit of the glucose dehydrogenase in an expressible form.

(4) The Escherichia bacterium according to (3), wherein the DNA encoding the γ-subunit locates upstream from the DNA encoding the α-subunit.

(5) The Escherichia bacterium according to any one of (1) to (4), wherein the Escherichia bacterium is Escherichia coli.

(6) A method for producing a glucose dehydrogenase complex, which comprises culturing the Escherichia bacterium according to any one of (1) to (5) so that the DNAs encoding the α-subunit and the β-subunit are expressed and the glucose dehydrogenase complex is produced and, collecting the complex.

### BRIEF DESCRIPTION OF THE DRAWINGS

FIG. 1 shows results of SDS-PAGE of GDH complexes purified from the Burkhorderia cepacia KS1 strain and the Escherichia coli JM109/pTrc99Aγαβ, pBBJMccm (photograph).

Lane 1: Marker

Lane 2: GDH purified from the KS1 strain

Lane 3: GDH purified from JM109/pTrc99Aγαβ, pBBJMccm

### BEST MODE FOR CARRYING OUT THE INVENTION

Hereafter, the present invention will be explained in detail.

The Escherichia bacterium of the present invention is an Escherichia bacterium into which DNAs encoding the α-subunit and the β-subunit (hereinafter, also referred to as “α-subunit gene” and “β-subunit gene”, respectively) of glucose dehydrogenase (hereinafter, also referred to simply as “GDH”) of Burkhorderia cepacia are introduced in an expressible form, and in which the ccm system is enhanced.

Examples of the aforementioned Escherichia bacterium include Escherichia coli.

Furthermore, examples of the aforementioned Burkhorderia cepacia include the KS1 strain, JCM2800 strain, JCM2801 strain, J2315 strain and so forth. The KS1 strain was deposited at the International Patent Organism Depositary, National Institute of Advanced Industrial Science and Technology (Tsukuba Central 6, 1-1, Higashi 1-Chome, Tsukuba-shi, Ibaraki-ken, 305-8566, Japan) and given an accession number FERM BP-7306. The JCM2800 strain and the JCM2801 strain are available from the Riken Bioresource Center, Japan Collection of Microorganisms (JCM). Further, the J2315 strain was deposited at the American Type Culture Collection (ATCC) with an ATCC number BAA-245 and the Belgian Co-ordinated Collections of Micro-organisms (BCCMTM) with an accession number LNG 16656, and is available from these depositories.

A nucleotide sequence of a chromosomal DNA fragment containing the α-subunit gene and a part of the β-subunit gene of GDH of the KS1 strain is shown in SEQ ID NO: 1 (refer to WO02/36779). Three open reading frames (ORFs) exist in this nucleotide sequence. The second and third ORFs from the 5′ end encode the α-subunit (SEQ ID NO: 3) and the β-subunit (SEQ ID NO: 4), respectively. Furthermore, the first ORF is estimated to encode the γ-subunit (SEQ ID NO: 2). A nucleotide sequence of a fragment containing the full-length β-subunit gene is shown in SEQ ID NO: 9. Furthermore, the amino acid sequence of the β-subunit is shown in SEQ ID NO: 10. In SEQ ID NO: 10, the amino acid numbers 1 to 22 are estimated to be a signal peptide. Although the first amino acid residue is indicated as Val in SEQ ID NOS: 9 and 10, it is very likely to be Met, and may be removed after translation.

The α-subunit gene used in the present invention is not limited to a gene encoding the amino acid sequence of SEQ ID NO: 3, and may be one encoding a protein having an amino acid sequence including substitution, deletion, insertion or addition of one or several amino acid residues in the amino acid sequence of SEQ ID NO: 3 so long as the encoded polypeptide has the GDH activity. Although an amino acid sequence that can be encoded by the nucleotide sequence of SEQ ID NO: 1 is shown in SEQ ID NO: 3, the methionine residue at the N-terminus may be removed after translation. The expression “one or several” mentioned above means preferably a number of 1 to 10, more preferably 1 to 5, particularly preferably 1 to 3.

Furthermore, the β-subunit gene may encode a protein having an amino acid sequence including substitution, deletion, insertion or addition of one or several amino acid residues in the amino acid sequence of the amino acid numbers 23 to 425 of SEQ ID NO: 10 so long as it functions as the β-subunit of GDH. The expression “one or several” mentioned above means preferably a number of 1 to 20, more preferably 1 to 10, particularly preferably 1 to 5. The expression “function as the β-subunit of GDH” means to function as cytochrome c without impairing the enzymatic activity of GDH.

Specific examples of the α-subunit gene include a DNA comprising the nucleotide sequence of the nucleotide numbers 764 to 2380 of SEQ ID NO: 1. Furthermore, the α-subunit gene may be a DNA hybridizable with a DNA comprising the nucleotide sequence of the nucleotide numbers 764 to 2380 of SEQ ID NO: 1 or a probe prepared from this sequence under stringent conditions and encoding a protein having the GDH activity.

Furthermore, specific examples of the β-subunit gene include a DNA comprising the nucleotide sequence of the nucleotide numbers 187 to 1398 of SEQ ID NO: 9. Furthermore, the β-subunit gene may be a DNA hybridizable with a DNA comprising the nucleotide sequence of the nucleotide numbers 187 to 1398 of SEQ ID NO: 9 or a probe prepared from this sequence under a stringent condition and encoding a protein that can function as the β-subunit.

The aforementioned stringent conditions include a condition under which DNAs having a homology of 70% or more, preferably 80% or more, more preferably 90% or more, hybridize with each other. Specifically, a condition of 1×SSC and 0.1% SDS at 60° C. can be mentioned.

The α-subunit gene and the β-subunit gene can be obtained by, for example, PCR using a chromosomal DNA of the Burkhorderia cepacia KS1 strain as a template. Primers for PCR can be prepared by chemical synthesis on the basis of the aforementioned nucleotide sequences. Furthermore, they can also be obtained from chromosomal DNA of the Burkhorderia cepacia KS1 strain by hybridization using an oligonucleotide prepared on the basis of the aforementioned sequences as a probe. Furthermore, their variants can also be obtained in the same manner from other Burkhorderia cepacia strains.

In the present invention, it is preferred that the α-subunit gene locates upstream from the β-subunit gene, and that their expressions are both regulated by a single promoter. Furthermore, it is preferred that a DNA encoding the γ-subunit (γ-subunit gene) is introduced into an Escherichia bacterium in an expressible form together with the α-subunit gene and the β-subunit gene. In this case, it is preferred that the γ-subunit gene locates upstream from the α-subunit gene, and that expressions of the subunit genes are all regulated by a single promoter.

A polycistronic DNA fragment encoding the γ-subunit, α-subunit and β-subunit in this order can be obtained by, for example, PCR using a chromosomal DNA of the Burkhorderia cepacia KS1 strain as a template and oligonucleotides having the nucleotide sequences of SEQ ID NOS: 12 and 13 as primers (see the examples described later).

To introduce a DNA containing the α-subunit gene and the β-subunit gene (and the γ-subunit gene as required) (hereinafter, referred to as “GDHαβ gene”) into an Escherichia bacterium in an expressible form, the GDHαβ gene can be inserted into a vector that functions in the Escherichia bacterium, and the Escherichia bacterium can be transformed with the obtained recombinant vector. In this case, by providing a promoter functioning in the Escherichia bacterium upstream from the GDHαβ gene, the GDHαβ gene can be expressed under the regulation by this promoter.

Examples of the vector that functions in an Escherichia bacterium include pBR322, pUC18, pUC118, pUC19, pUC119, pACYC184, pBBR122 and so forth. Furthermore, examples of the aforementioned promoter include lac, trp, tac, trc, P_{L}, tet, PhoA and so forth. Furthermore, by inserting the GDHαβ gene into an expression vector containing a promoter at a suitable site, the insertion of the gene into the vector and the ligation of the promoter can be performed in the same step. Examples of such an expression vector include pTrc99A, pBluescript, pKK223-3 and so forth.

Furthermore, the GDHαβ gene may be incorporated into a chromosomal DNA of an Escherichia bacterium in an expressible form.

To transform an Escherichia bacterium with a recombinant vector, the competent cell method using a calcium treatment, electroporation or the like can be employed, for example.

The Escherichia bacterium of the present invention is an Escherichia bacterium which is introduced with the GDHαβ gene as described above, and in which expression of the ccm system is enhanced.

The ccm system is encoded by the ccm operon (ccmABCDEFGH). The ccm operon can be obtained by, for example, PCR using a chromosomal DNA of Escherichia coli as a template. Primers for PCR can be prepared by synthesis on the basis of the reported nucleotide sequence (DDBJ/EMBL/GenBank accession number AE005452). Furthermore, they can be obtained from a chromosomal DNA of Escherichia coil by hybridization using an oligonucleotide prepared on the basis of the aforementioned sequence as a probe. Furthermore, their variants can also be obtained in the same manner from other Escherichia bacteria.

The ccm operon may be, in addition to the DNA having the aforementioned reported nucleotide sequence, a DNA hybridizable with the DNA comprising the nucleotide sequence or a probe prepared from the sequence under stringent conditions and encoding a group of enzymes functioning as the ccm system.

The ccm system is known to be expressed under anaerobic and special conditions (non-patent document 1). In the present invention, the expression “expression of the ccm system is enhanced” means that the expression is enhanced compared with that in a wild strain or unmodified strain of Escherichia bacteria, or that a modification is made so that the system can be expressed even under conditions which are not the anaerobic and special conditions. Examples of conditions which are not the anaerobic and special conditions include an aerobic condition.

To enhance the expression of the ccm system, genes of the ccm operon can be ligated to a promoter that constitutively expresses them or a promoter that can regulate their expressions, and the obtained recombinant gene can be introduced into an Escherichia bacterium. Preferred promoters and vectors, and introduction of the ccm operon into an Escherichia bacterium are similar to those described with regard to the GDHαβ gene.

Examples of a plasmid containing the ccm operon include pEC86 obtained by inserting the operon into pACYC184. This operon is constitutively expressed under regulation by the tet promoter. Furthermore, a plasmid into which the ccm operon cloned by PCR or the like from a chromosomal DNA of Escherichia bacterium is inserted so as to be under control of a suitable promoter can also be prepared.

Furthermore, the expression of the operon can also be enhanced by replacing the promoter of the ccm operon on a chromosomal DNA of Escherichia bacterium with a suitable promoter.

The GDH complex can be efficiently produced by culturing the Escherichia bacterium of the present invention to allow the bacterium to express the α-subunit gene and the β-subunit gene and produce the GDH enzyme complex as an expression product of these and collecting the complex. The term “GDH complex” used here preferably means a multimeric protein formed by association of the subunits, but also includes a mixture of the subunits in free forms.

As for the method of culturing the Escherichia bacterium, culture conditions can be selected taking nutritional and physiological properties of a host into consideration. The culture is performed as liquid culture in many cases. Industrial culture is advantageously performed with aeration by stirring.

As the nutrients of the medium, those usually used for culture of Escherichia bacteria can be extensively used. As the carbon source, a carbon compound that can be assimilated may be used, and for example, glucose, sucrose, lactose, maltose, galactose, molasses, pyruvic acid and so forth are used. As the nitrogen source, a nitrogen compound that may be utilized, and for example, peptone, meat extract, yeast extract, casein hydrolysate, soybean meal alkali extract and so forth are used. In addition, phosphoric acid salts, carbonic acid salts, sulfuric acid salts, salts of magnesium, calcium, potassium, iron, manganese, zinc and so forth, specific amino acids, specific vitamins and so forth are used as required.

The culture temperature can be suitably changed within the range in which Escherichia bacteria grow and produce the GDH complex, and is preferably about 20 to 42° C. Although the culture time slightly varies depending on the conditions, it is sufficient that culture is terminated at an appropriate time by estimating the time when the maximum GDH complex yield is attained, and the culture time is usually about 12 to 72 hours. pH of the medium can be suitably changed within the range in which bacterial cells grow and produce the GDH complex, and is preferably within the range of about pH 6.0 to 9.0.

As the GDH complex, the culture broth as it is may be collected and used. However, when the GDH complex exists in the culture broth, it is generally used as a solution containing the GDH complex after separation from Escherichia bacterium cells by filtration, centrifugation or the like in a conventional manner. When the GDH complex exists in cells, it is isolated and collected as an aqueous solution by collecting the cells from the obtained culture by means of filtration, centrifugation or the like, then disrupting the cells by a physical method or an enzymatic method using lysozyme or the like, and adding a chelating agent such as EDTA and a surfactant, if necessary, to solubilize the subunits.

The GDH complex can be precipitated from the solution obtained as described above by, for example, vacuum concentration, membrane concentration, salting out using ammonium sulfate, sodium sulfate or the like, or fractional precipitation using a hydrophilic organic solvent such as methanol, ethanol or acetone. Furthermore, heat treatment and isoelectric precipitation are also effective purification means. Then, purification can be performed by a suitable combination of gel filtration using an adsorbent, gel filtration agent or the like, absorption chromatography, ion exchange chromatography, affinity chromatography and so forth to obtain a purified GDH complex. A purified enzyme preparation can be obtained by isolation and purification based on column chromatography.

The α-subunit of GDH solely exhibits the enzymatic activity. Therefore, the α-subunit alone can be isolated and purified from the Escherichia bacterium or the GDH complex of the present invention and used.

### EXAMPLES

The present invention will be explained more specifically with reference to the following examples.

### Example 1

### Isolation of Gene Encoding GDH β-Subunit of Burkhorderia cepacia KS1 Strain

<1> Search of GDH β-Subunit of Burkhorderia cepacia KS1 Strain

The GDH β-subunit gene derived from the KS1 strain was searched by using the Burkhorderia cepacia J2315 strain genome database of the Sanger Centre (http://www.sanger.ac.uk/). With reference to the already elucidated N-terminus sequence of the GDH β-subunit of the KS1 strain (SEQ ID NO: 5), an amino acid sequence (SEQ ID NO: 6) having a homology to the cytochrome c subunits of alcohol dehydrogenases derived from Acetobacter sp. and Gluconobacter sp. (Tamaki T. et al., Biochem. Biophys. Acta, 1088(2): 292-300 (1991); Matsushita K. et al., Biosci. Biotech. Biochem., 56, 304-310 (1992); Takemura H. et al., J. Bacteriol., 175, 6857-66 (1993); Kondo K. et al., Appl. Environ. Microbiol., 63, 1131-8 (1997)), gluconate dehydrogenases derived from Erwinia sp. and Pseudomonas sp. (Yum D. Y. et al., J. Bacteriol., 179, 6566-72, (1997); Matsushita K. et al., J. Biochem., 85, 1173-81 (1979)), sorbitol dehydrogenase derived from Gluconobacter sp. (Choi E. S. et al., FEMS Microbiol. Lett., 125, 45-50 (1995)) and 2-ketogluconate dehydrogenases derived from Erwinia sp. and Pantoea sp. (Pujol C. J. et al., J. Bacteriol., 182, 2230-7 (2000)) was designed.

Using the aforementioned amino acid sequence as an index, gene sequences encoding amino acid sequences showing a high homology were searched from the aforementioned database of Burkhorderia cepacia J2315 strain using BLAST. Then, five of the obtained sequences were examined for homology to the C-terminus sequence of the GDH α-subunit of the KS1 strain. As a result, amino acid sequences translated from two of gene fragments showed a high homology (>90%). Because each gene fragment was as short as 200 to 500 bp, sequences showing a high homology with these sequences were searched from the genome database of the Burkhorderia cepacia J2315 strain by using BLAST, and the fragments were joined. As a result, a fragment of 3110 bp was obtained. In the obtained nucleotide sequence, an ORF that appeared to be the one for the C-terminus of GDH and an ORF of 1275 bp that appeared to be the structural gene of cytochrome c existed. The obtained nucleotide sequence of the J2315 strain and the already cloned nucleotide sequence of the KS1 strain α-subunit were compared. The result showed that a nucleotide sequence showing a high homology to the nucleotide sequence encoding the signal peptide of J2315 strain cytochrome c was contained downstream from the α-subunit.

From the above, the third ORF (the nucleotide numbers 2386 and the followings in SEQ ID NO: 1) in the already cloned GDH gene of the Burkhorderia cepacia KS1 strain (SEQ ID NO: 1, refer to WO 02/36779) was estimated to encode the β-subunit. Furthermore, because the amino acid sequence at the N-terminus of the purified β-subunit matched 5 amino acid residues translated from the nucleotide sequence of the nucleotide numbers 2452 to 2466 in SEQ ID NO: 1, the aforementioned ORF was considered to encode the β-subunit.

<2>

Amplification of β-Subunit Structural Gene by Inverse PCR

(1) Culture of Cells and Extraction of Genome

The KS1 strain was cultured overnight at 37° C. in 5 ml of a complete medium (0.5% polypepton, 0.3% yeast extract, 0.5% NaCl) with shaking. The genome was extracted from the obtained cells using GennomicPrep™ Cells and Tissue DNA Isolation Kit (Amersham Pharmacia Biotech). The procedure was performed according to the attached manual. The obtained genome was subjected to a phenol/chloroform treatment and ethanol precipitation, and then dissolved in purified water.

(2) Cyclization of Genome Fragments

The genome extracted from the KS1 strain was digested with BamHI, EcoRI, HindIII, SmaI, SacI and XhoI, and the genome fragments were collected by ethanol precipitation. A ligation reaction was performed overnight at 16° C. for 1 μg of the genome digested with the restriction enzymes by using DNA Ligation Kit (Takara Shuzo).

(3) PCR

In an amount of 50 pmol of a forward primer (EF1, SEQ ID NO: 7) and 50 pmol of a reverse primer (ER1, SEQ ID NO: 8) designed on the basis of the nucleotide sequence of the N-terminus signal sequence region of the GDH β-subunit of the KS1 strain (both the primers were synthesized by Invitrogen on commission), 0.5 ml of LATaq (Takara Bio Inc.), 8 μl of dNTP solution and 5 μl of 10× PCR buffer were added with purified water to make the total volume of 50 μl, and PCR was performed using Program Temp Control System PC-801 (ASTEC). PCR was performed under the following condition. A cycle consisting of reactions at 94° C. for 5 minutes, at 98° C. for 20 seconds and at 62° C. for 30 seconds was repeated 30 times, and followed by reactions at 72° C. for 6 minutes and at 72° C. for 10 minutes.

When the genome digested with the restriction enzyme SmaI was used as a template, a fragment of about 2.1 kbp was confirmed by agarose electrophoresis.

<3> Sequencing of PCR-Amplified Fragment

(1) TA Cloning

The aforementioned inverse PCR product was subjected to agarose gel electrophoresis, and a band was excised and purified by using Gene Clean II Kit (Bio101 Inc.). This fragment was ligated to the pGEM-T vector by using pGEMR-T and pGEMR-T EASY Vector Systems (Promega). Escherichia coli DH5α was transformed with the ligated vector and cultured overnight in an L agar medium containing 50 μg/ml of ampicillin, 40 μg/ml of X-Gal and 0.1 μM IPTG. A white colony was selected among the colonies that emerged and cultured overnight in an L medium containing 50 μg/ml of ampicillin, and plasmids were extracted from the cells by the alkali method.

(2) Preparation of Sequence Sample

The obtained plasmids were subjected to an RNase treatment, added with 20% PEG6000/2.5 M NaCl in a volume of 0.6 times the plasmids and left on ice for 1 hour. Then, the mixture was centrifuged at 15000 rpm at 4° C. for 15 minutes to obtain a pellet. The pellet was washed with 70% ethanol, vacuum dried, and dissolved in purified water.

(3) Analysis of DNA Nucleotide Sequence

The nucleotide sequence of the fragment inserted into the plasmid obtained in (2) was analyzed by using ABI PRISM™ 310 Genetic Analyzer (PERKIN-ELMER Applied Biosystems). A sequence of a part of the inserted fragment from the multi-cloning site of the vector was determined by using the M13 primer. As a result, a nucleotide sequence including the already elucidated sequence for the N-terminus of the β-subunit was confirmed. Primers were successively prepared on the basis of this sequence and used to determine the nucleotide sequence of the inserted fragment. The result is shown in SEQ ID NO: 9. Furthermore, the amino acid sequence encoded by the ORF included in this nucleotide sequence is shown in SEQ ID NO: 10.

The β-subunit consists of 425 amino acid residues in total. Among these, 22 residues are considered to be a signal peptide on the basis of comparison with the already obtained N-terminus amino acid sequence. The molecular weight calculated from the amino acid sequence was 45,276 Da, and the molecular weight excluding the signal peptide, 42,731 Da, was equivalent to the molecular weight of GDH β-subunit of the KS1 strain obtained by SDS-PAGE, 43 kDa. The heme binding motif (SEQ ID NO: 11) was confirmed at 3 sites in cytochrome c in the amino acid sequence of the β-subunit. This ORF located immediately downstream from the ORF of the α-subunit structural gene, and a sequence that appeared to be the SD sequence existed upstream from the start codon.

A homology search was performed for this amino acid sequence by using BLAST, and an overall high homology was observed at the amino acid level, i.e., a homology of 65% to the cytochrome c subunit of oxide reductase dehydrogenase derived from Ralstonia solanacearum, 48% to the cytochrome c subunit of sorbitol dehydrogenase derived from Gluconobacter oxydans, 44% to the cytochrome c subunit of gluconate dehydrogenase derived from Eriwinia cypripedii and 46.4% to the cytochrome c subunit of 2-ketogluconate dehydrogenase derived from Pantoea citrea. Furthermore, the heme binding motif sequence (SEQ ID NO: 11) was conserved in these amino acid sequences of cytochrome c.

The GDH β-subunit structural gene of the KS1 strain has homologies of 92.0% and 92.2% to the GDH β-subunit structural gene of the J2315 strain at the nucleotide sequence level and the amino acid level, respectively.

### Example 2

### Introduction of GDHαβ Gene into Escherichia coli and Enhancement of ccm System

<1> Preparation of Chromosomal DNA from Burkhorderia cepacia KS1 Strain

Chromosomal genes were prepared from the Burkhorderia cepacia KS1 strain in a conventional manner. That is, the bacterial strain was cultured overnight in a TL liquid medium (10 g of polypeptone, 1 g of yeast extract, 5 g of NaCl, 2 g of KH₂PO₄, 5 g of glucose in 1 L, pH 7.2) at 34° C. with shaking. The proliferated cells were collected by centrifugation. The cells were suspended and treated at 50° C. for 6 hours in a solution containing 10 mM NaCl, 20 mM Tris-HCl (pH 8.0), 1 mM EDTA, 0.5% SDS and 100 μg/ml of proteinase K. This suspension was added with an equivalent volume of phenol/chloroform, stirred at room temperature for 10 minutes and centrifuged to obtain a supernatant. This supernatant was added with sodium acetate to a final concentration of 0.3 M and overlaid with ethanol in a volume twice the volume of the supernatant to precipitate chromosomal DNA in the intermediate layer. It was scooped with a glass rod, washed with 70% ethanol and dissolved in a suitable volume of TE buffer to obtain a chromosomal DNA solution.

<2> Preparation of GDHαβ Gene

DNA fragments encoding the γ-subunit, the α-subunit and the β-subunit of GDH were amplified by PCR using the aforementioned chromosomal DNA as a template and oligonucleotides having the following sequences as primers.

<Forward primer: cF1>

(SEQ ID NO: 12)

5′-CATGCCATGGCACACAACGACAACAC-3′
<Reverse primer: GDHbU1>

(SEQ ID NO: 13)

5′-GTCGACGATCTTCTTCCAGCCGAACATCAC-3′

The C-terminus side of the amplified fragment was blunt-ended, then the N-terminus side was digested with NcoI, and the fragment was ligated to the similarly treated pTrc99A (Pharmacia). E. coli DH5α was transformed with the obtained recombinant vector, and a colony that emerged on an LB agar medium containing 50 μg/mL of ampicillin was collected. The obtained transformant was cultured in a liquid LB medium to extract the plasmids. When the inserted DNA fragment was analyzed, an inserted fragment of about 3.8 kb was confirmed. This plasmid was designated pTrc99Aγαβ. The GDHαβ gene in this plasmid is regulated by the trc promoter. pTrc99Aγαβ contains the ampicillin resistance gene.

<3> Preparation of ccm System Plasmid

pTrc99Aγαβ was digested with EcoT22I, and then the end was blunt-ended. Then, the fragment was digested with NotI and subjected to agarose gel electrophoresis to isolate and collect a short DNA fragment. This DNA fragment was inserted into pBBR122 digested with ScaI and NotI (MoBiTec) to prepare pBBGDHγαβ.

Chromosomal genes were prepared from E. coli JM109 in a conventional manner. That is, this bacterial strain was cultured overnight at 37° C. in an LB medium (10 g of polypeptone, 5 g of yeast extract, 10 g of NaCl in 1 L, pH 7.0) with shaking. The proliferated cells were collected by centrifugation. The cells were suspended and treated at 50° C. for 6 hours in a solution containing 10 mM NaCl, 20 mM Tris-HCl (pH 8.0), 1 mM EDTA, 0.5% SDS and 100 μg/ml of proteinase K. This suspension was added with an equivalent volume of phenol/chloroform, stirred at room temperature for 10 minutes and centrifuged to obtain a supernatant. This supernatant was added with sodium acetate to a final concentration of 0.3 M and overlaid with ethanol in a volume twice the volume of the supernatant to precipitate chromosomal DNA in the intermediate layer. It was scooped with a glass rod, washed with 70% ethanol and dissolved in a suitable volume of TE buffer to obtain a JM109 chromosomal DNA solution.

The DNA fragment encoding the ccm system gene was amplified by PCR using the JM109 chromosomal DNA as a template and oligonucleotides having the following sequences as primers.

<Forward primer: ECccmD1>

(SEQ ID NO: 14)

5′-TGGCCATGGTTGAAGCCAGAGAGTTACTTT-3′
<Reverse primer: ECccmU1>

(SEQ ID NO: 15)

5′-TTATTTACTCTCCTGCGGCGACAAATGTTG-3′

The amplified end was blunt-ended, and then the N-terminus side was digested with NcoI. This fragment was digested with ACCI, blunt-ended, and then ligated to pBBGDHγαβ digested with NcoI to prepare pBBJMccm. The GDHαβ gene was removed by the digestion with AccI, blunt-ending and the subsequent digestion with NcoI of pBBGDHγαβ.

<4> Introduction of GDHαβ Gene and ccm System into Escherichia coli

E. coli JM109 was transformed with pTrc99Aγαβ and pBBJMccm to obtain JM109/pTrc99Aγαβ, pBBJMccm. Furthermore, as a control, E. coli JM109 was transformed with pTrc99Aγαβ to obtain JM109/pTrc99Aγαβ.

These transformants were cultured overnight in 10 mL of 2× YT medium containing 50 μg/ml of ampicillin and 50 μg/ml of kanamycin (JM109/pTrc99Aγαβ, pBBJMccm), or 50 μg/ml of ampicillin (JM109/pTrc99Aγαβ) at 34° C. with shaking. Furthermore, the Burkhorderia cepacia KS1 strain was cultured overnight in 10 mL of a complete medium with shaking. The cells were collected from a part of each culture broth by centrifugation, added with 10 mM potassium phosphate buffer (pH 7.0) containing 1% sodium cholate to the volume before the centrifugation and then disrupted by ultrasonication. Then, the GDH activity in the supernatant obtained by centrifuging the disrupted suspension was determined.

The GDH activity was determined by the following procedure. A mixture of 47 mM phosphate buffer (pH 6.0), 20 mM glucose, 2 mM phenazine methosulfate and 0.1 mM 2,6-dichlorophenol indophenol was heated at 37° C. beforehand and added to a sample to start a reaction. While the mixture was maintained at 37° C., changes in absorbance at 600 nm were measured to determine the activity. The GDH activity was obtained by using a molecular extinction coefficient (4.76 mM/cm) of 2,6-dichlorophenol indophenol. One unit (U) of the enzymatic activity was defined as the oxidization amount of 1 μmol of 2,6-dichlorophenol indophenol per minute.

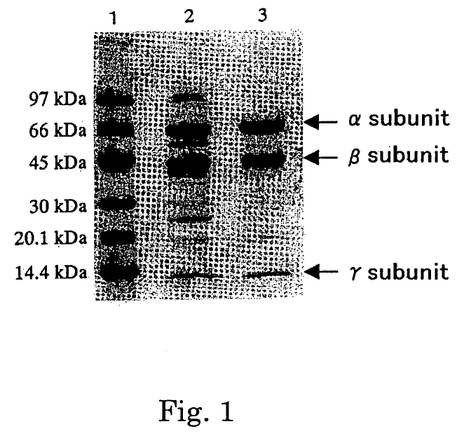

As a result, GDH activities of JM109/pTrc99Aγαβ, Burkhorderia cepacia KS1 and JM109/pTrc99Aγαβ, pBBJMccm were 0.3, 1.4 and 32 U/mL, respectively. That is, JM109/pTrc99Aγαβ hardly exhibited the GDH activity, and Burkhorderia cepacia KS1, a wild strain, exhibited a weak GDH activity. However, JM109/pTrc99Aγαβ, pBBJMccm exhibited an extremely high GDH activity. In addition, the GDH complex was purified from JM109/pTrc99Aγαβ, pBBJMccm and subjected to SDS-PAGE. As a result, it could be confirmed that the GDH complex showed the same electrophoresis pattern as that of the GDH complex purified from the KS1 strain (FIG. 1).

### INDUSTRIAL APPLICABILITY

According to the present invention, an enzyme complex containing the α-subunit and the β-subunit of glucose dehydrogenase of Burkhorderia cepacia can be abundantly expressed in an Escherichia bacterium.

1. An Escherichia bacterium, comprising DNAs encoding the α-subunit and the β-subunit of glucose dehydrogenase of Burkhorderia cepacia in an expressible form, wherein the bacterium is further modified so that the expression of a ccm system is enhanced.

2. The Escherichia bacterium according to claim 1, wherein the DNA encoding the α-subunit is located upstream from the DNA encoding the β-subunit, and expression of the subunits is regulated by a single promoter.

3. The Escherichia bacterium according to claim 1, further comprising a DNA encoding the γ-subunit of glucose dehydrogenase in an expressible form.

4. The Escherichia bacterium according to claim 3, wherein the DNA encoding the γ-subunit is located upstream from the DNA encoding the α-subunit.

5. The Escherichia bacterium according to claim 1, wherein the Escherichia bacterium is Escherichia coli.

6. A method for producing a glucose dehydrogenase complex, which comprises culturing the Escherichia bacterium according to claim 1 so that the DNAs encoding the α-subunit and the β-subunit are expressed and the glucose dehydrogenase complex is produced, and collecting the complex.

7. The Escherichia bacterium according to claim 1, wherein the bacterium is modified so that the expression of the ccm system is enhanced by the bacterium, comprising a plasmid comprising genes of a ccm operon operably linked to a promoter.

8. The Escherichia bacterium according to claim 7, wherein the plasmid is pEC86.

9. The Escherichia bacterium according to claim 1, wherein the bacterium is modified so that the expression of the ccm system is enhanced by replacing the bacterium's ccm operon promoter with another promoter.
